(19) United States
(12) Patent Application Publication
(10) Pub. No.: US 2004/0265813 A1
(43) Pub. Date: Dec. 30, 2004

(54) Dna arrays for measuring sensitivity to anticancer agent

(76) Inventors: Teiji Takechi, Tokorozawa-shi (JP); Katsuhisa Koizumi, Hanno-shi (JP); Atsushi Azuma, Tokorazawa-shi (JP); Masakazu Fukushima, Hanno-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/482,158

(22) PCT Filed: Jul. 3, 2002
(86) PCT No.: PCT/JP02/06754

(30) Foreign Application Priority Data
Jul. 5, 2001 (JP) 2001-204448
Aug. 7, 2001 (JP) 2001-239181

(51) Int. Cl. C12Q 1/68; C12M 1/34

(52) U.S. Cl. 435/6; 435/287.2

(57) ABSTRACT

A DNA array for measuring sensitivity to an antimetabolite-type anticancer agent or a combined use of such an anticancer agent and another anticancer agent, characterized by including at least 13 types of target gene fragments, involving at least two types of genes selected from each of the following groups: nucleic acid metabolism-associated enzyme genes, gene repair-associated enzyme genes, drug resistance-associated factor genes and housekeeping genes, wherein these gene fragments have been selected by the following steps 1) and 2) and immobilized on a substrate; 1) a step of selecting fragments having high specificity for target genes by searching the homology with the use of databases; and 2) a step of performing Northern hybridization against RNA obtained from tumor cells with the use of the fragments selected in the step 1) as probes to thereby confirm the specificity for the target genes. Thus, through a single assay procedure, expression of several tens to several hundreds of genes in a specimen can be conveniently measured at a high quantitation level.

### TECHNICAL FIELD

The present invention relates to a DNA array for measuring sensitivity to an anticancer agent, and to a method for measuring such sensitivity by use of the DNA array.

### BACKGROUND ART

Generally speaking, anticancer agents are not necessarily satisfactory in terms of therapeutic effects on cancer patients, permitting relatively high incidences of adverse side effects. In recent years, demand has arisen for proper use of anticancer agents and treatment that is custom-tailored to the conditions of the individual patient, wherein before administration of an anticancer agent to a patient, a tissue specimen or a similar specimen is collected from the patient, and while the specimen is used as a test sample, the expression level of a gene associated with sensitivity to the anticancer agent is measured. This allows proper selection of patients who would be expected to exhibit satisfactory effect of the drug or a low level of adverse side effect.

Clinical research efforts have been devoted toward establishing criteria for proper use of 5-fluorouracil (5-FU) agents and other antimetabolites among popular anticancer agents. Typical target genes for serving assay are genes of thymidylate synthase (EC2.1.1.45, hereinafter referred to as “TS”), dihydropyrimidine dehydrogenase (EC1.3.1.2, hereinafter referred to as “DPD”), and thymidine phosphorylase (EC2.4.2.4, hereinafter referred to as “TP”), which are considered to be associated with sensitivity to this antimetabolite. Expression patterns of these genes or enzymes are analyzed through biochemical assays for determining enzymatic activity (Clinical Cancer Research, 5, 883-889, 1999), immunoassays using antibodies (Cancer and Chemotherapy, 24(6): 705-712, 1997), or mRNA assays making use of Northern hybridization or RT-PCR (Clinical Cancer Research, 6, 1322-1327, 2000), and the results therefrom are used as indices of proper use. In cancer chemotherapy using cisplatin, DNA repair enzymes such as ERCC1 are suggested to be useful as factors that predict the effect of therapy (Journal of Clinical Oncology, 16, 309-316, 1998).

Meanwhile, the DNA microarray method has recently come into widely use, as it enables simultaneous analysis of mRNA expression of several hundred to some dozens of thousands of diversified genes. This technique is suitable for comprehensive analysis of genes, and thus is expected to be applied to realization of proper use of anticancer agents in the future.

In the human body, 5-fluorouracil and other antimetabolites are metabolized through a variety of processes. Therefore, only the gene expression analysis of the above-mentioned 3 types of genes (TS, DPD, and TP) is not sufficient for predicting sensitivity to antimetabolites. If gene expression can be analyzed in broader ranges, sensitivity would be better predicted. In actual settings of cancer chemotherapies, combination therapy is commonly performed, in which several types of anticancer agents are used in combination. In such cases, if expression of genes that are associated with sensitivity to each drug can be analyzed comprehensively, suitable combination chemotherapy can be designed for each individual patient.

Biochemical assays for measuring enzyme activity, immunoassays making use of antibodies, Northern hybridization, and RT-PCR mRNA assays provide quantitatively precise data. However, in principle, they require one assay for each gene, and therefore, they are not suitable for simultaneous analysis of diversified modes of gene expression.

In contrast, the DNA microarray method is useful for the analysis of diversified gene expressions. However, this method is far inferior to Northern hybridization or RT-PCR in terms of quantitative preciseness. One of the main reasons may be explained as follows: Northern hybridization and RT-PCR can designate, based on the size of RNA or PCR product, the specificity to a specific gene of interest, whereas the DNA microarray method cannot, because this method relies on dot blots which do not show the molecular size, leading to false detection; i.e., detection of non-specific signals attributed to expression of genes other than the gene of interest (so-called cross-hybridization). Specificity to a gene of interest is affected by the selection of DNA fragments to be arrayed (hereinafter such DNA fragments will be referred to as the target fragments); i.e., selection of portions (regions) of the gene (full-length cDNA) as target fragments. Specificity obtained in an ordinarily performed DNA microarray method has not yet been substantiated, although the target fragments employed in the method are determined among the regions that have been predicted to have high specificity from computation (regions showing minor nucleotide sequence overlapping with corresponding sequences of other genes which are publicly available from databases) by use of design assistance software. Moreover, primers which have specificity to each individual gene employed in PCR, which is performed for preparing target fragments through amplification, may migrate to a target fragment solution and be included in arrays, raising the risk of causing non-uniform background. From these factors, even for a single specimen (a sample derived form a living organism), expression level of a specific gene analyzed through the DNA microarray method often disagrees with the results obtained through Northern hybridization or RT-PCR, and therefore, it is improper to conclude existence of a difference in gene expression solely from the results obtained from the DNA microarray method. According to a generally employed test procedure, genes which are likely to show different levels of expression are roughly identified through a first screening by use of the DNA microarray method, and then, through a second screening, like Northern hybridization or RT-PCR is performed to verify that differences in expression level in fact exist. Thus, in such a scheme, at least 2 assay methods must be employed.

Accordingly, an object of the present invention is to provide means for measuring, in single measurement, sensitivity to an antimetabolite-type anticancer agent or to a combination of such an anticancer agent and another anticancer agent through a simple assay with high sensitivity and better quantitativeness.

### DISCLOSURE OF THE INVENTION

Under the above circumstances, the present inventors have studied whether sensitivity to an anticancer agent can be determined through a single measurement by use of the widely employed existing DNA microarray method, and have identified the following problems.

Briefly, in use of the widely employed DNA microarray method, since the species of potential target genes counts several hundred to several dozens of thousands, it is considered to include those genes that are irrelevant to sensitivity to drugs, as conjectured from action mechanisms of currently available anticancer agents. As a result, analysis would require extra work, or genes which are closely related to the mechanisms of action of the anticancer agent may be overlooked or given less weight than they should be.

Moreover, in the case of the widely employed DNA microarray method, the quantity (concentration) of DNA to be spotted on a support carrier (e.g., nylon membrane, glass plate) is usually prefixed, and therefore, quantification may sometimes be unsuccessful because of extremely low expression level, despite the gene being closely related to sensitivity to the anticancer agent.

Thus, the present inventors have set out to improve the existing DNA microarray method. Firstly, they have narrowed the range of target genes to dozens to several hundreds of genes centering on those which are considered to be associated with mechanisms of action of an antimetabolite-type anticancer agents and other anticancer agents to be used in combination therewith. Next, through a homology-based search performed using databases, not only those genes determined through calculation but also those actually confirmed to have specificity through Northern hybridization, are determined to be target fragments. The thus-created DNA array in which fragments selected as described above are immobilized on the substrate has been found to be capable of providing judgments, by a single test with high sensitivity, with respect to an antimetabolite-type anticancer agent or a combination use of such an anticancer agent and another anticancer agent, thus leading to the present invention.

Accordingly, the present invention provides a DNA array for measuring sensitivity to an antimetabolite-type anticancer agent or to a combination of such an anticancer agent and another anticancer agent, characterized by comprising a substrate and target gene fragments attached to the substrate, the target gene fragments being of at least 13 gene species, including at least 2 species from each of the following gene groups: a group of genes coding for nucleic-acid-metabolism-related enzymes, a group of genes coding for gene-repair-associated enzymes, a group of genes coding for drug resistance-associated factors, and housekeeping genes, and said at least 13 gene species being selected by performing the following two steps 1) and 2):

1) selecting fragments having high target-gene specificity through a homology search using a database, and

2) performing Northern hybridization to RNA collected from tumor cells, using the fragments selected in step 1) as probes, to thereby confirm target-gene specificity.

The present invention also provides a method for measuring sensitivity of a body fluid specimen or tissue specimen of a cancer patient to an antimetabolite-type anticancer agent or to a combination of such an anticancer agent and another anticancer agent, characterized by comprising hybridizing the DNA array with labeled cDNA probes synthesized through use, as a template, of mRNA obtained from the specimen.

### BRIEF DESCRIPTION OF THE DRAWINGS

FIG. 1 shows universal primer sequences and the structure of a clone to which a target fragment is incorporated.

FIG. 2 contains actual images obtained from Northern hybridization, showing that in some cases different specificities result when DNA fragments serving as templates in the synthesis of probes are determined in different regions of a gene.

FIG. 3 shows the correlation between total RNA quantity and the measurements obtained in Northern hybridization.

FIG. 4 contains charts showing diagnosis quality provided by the array of the present invention, assessed in comparison with Northern hybridization.

FIG. 5 shows the correlation between TS-1 sensitivity and expression level, found in 11 types of xenografts, and contains graphs in connection with four genes which among 52 types of genes exhibited higher correlation.

FIG. 6 shows the correlation between TS-1 sensitivity and TS expression level, found in the 11 types of xenografts.

### BEST MODE FOR CARRYING OUT THE INVENTION

The DNA array of the present invention is contemplated to be used in determining sensitivity to an antimetabolite serving as an anticancer agent (may be referred to as an antimetabolite-type anticancer agent), or in determining sensitivity to a combination of such an anticancer agent and another anticancer agent. Examples of the antimetabolite-type anticancer agent include 5-fluorouracil anticancer agents such as tegafur, 5-fluorouracil, tegafur.uracil, tegafur.gimeracil.oteracil potassium, carmofur, capecitabine, and furtulon; mercaptopurine anticancer agents such as 6-mercaptopurine and mercaptopurine riboside; anticancer cytosine analogues such as cytarabine, enocitabine, and gemcitabine; and methotrexate. Of these, 5-fluorouracil anticancer agents are particularly preferred. Examples of the mentioned “another anticancer agent” to be used in combination with the antimetabolites include platinum -complex-based anticancer agents such as cisplatin; topoisomerase inhibitors such as CPT-11 and VP-16; and taxane anticancer agents such as docetaxel and paclitaxel.

The measurement of sensitivity, as used in the present invention, refers to determination or judgment in terms of balance between efficacy of the anticancer agent on a patient and adverse side effects; decision of appropriate combination therapy, determination of appropriate administration scheme (dose, drug administration regimen), etc.

The target genes of the present invention are those which are considered to be associated with sensitivity, and consist of at least 13 species of gene, including at least two different species from each of the following groups: a group of genes coding for nucleic acid metabolism-related enzymes, a group of genes coding for gene-repair-associated enzymes, a group of genes coding for drug resistance-related factors, and a group of housekeeping genes.

Examples of the nucleic acid metabolism-related enzymes include thymidylate synthase (TS), dihydropyrimidine dehydrogenase (DPD), orotate phosphoribosyltransferase (OPRT) (uridine monophosphate synthetase (UMPS)), thymidine phosphorylase (TP), thymidine kinase 1 (TK1), ribonucleoside-diphosphate reductase M1 subunit (RRM1), ribonucleoside-diphosphate reductase M2 subunit (RRM2), uridine cytidine kinase 2 (UCK2), uridine phosphorylase (UP), cytidine deaminase (CDA), 5′nucleotidase (NT5), IMP dehydrogenase 1 (IMPD), methylenetetrahydrofolate dehydrogenase (MTHFD1), RNA polymerase 2 (RP2), uridine monophosphate kinase (UMPK), CTP synthase (CTPS), deoxycytidylate deaminase (DCD), deoxycytidine kinase (DCK), phosphoribosyl pyrophosphate synthetase (PRPS), hypoxanthine phosphoribosyltransferase 1 (HPRT1), folylpolyglutamate synthetase (FPGS), nucleoside diphosphate kinase A (NDKA), nucleoside diphosphate kinase B (NDKB), adenine phosphoribosyltransferase (APRT), and adenosine kinase (AK). Among the genes coding for these enzymes, preferably, at least genes coding for TS, DPD, ORRT, TP, and TK1 are employed.

Examples of the gene-repair-associated enzymes include DNA excision repair protein ERCC1 (ERCC1), uracil-DNA glycosylase (UDG), poly(ADP-ribose) polymerase (PARP), DNA ligase I (LIG1), DNA ligase III (LIG3), DNA ligase IV (LIG4), DNA polymerase β (POLB), DNA polymerase δ (POLD), and DNA-repair protein XRCC1 (XRCC1). Among the genes coding for these enzymes, preferably, at least genes coding for ERCC1 and UDG are employed.

Examples of drug resistance-related factors include topoisomerase 1 (TOP1), P-glycoprotein (MDR1), equilibrative nucleoside transporter 1 (ENT1), multidrug resistance-associated protein 1 (MRP1), topoisomerase 2α (TOP2A), topoisomerase 2β (TOPB), heatshockprotein 27 (Hsp27), and equilibrative nucleoside transporter 2 (ENT2). Among the genes coding for these factors, preferably, at least genes coding for TOP1, MDR1, ENT1, and MRP1 are employed.

Examples of the housekeeping genes include genes coding for glyceraldehyde-3-phosphate dehydrogenase (GAPDH), β-actin (ACTB), and 40S ribosomal protein S9 (RSP9). Preferably, at least two out of GAPDH gene, ACTB gene, and RSP9 gene are employed. These genes are used as internal standards.

Example of other genes include those coding for E2F1, p53, VEGF β, integrin α3, Mn SOD, Cu/Zn SOD, or proliferating cell nuclear antigen (PCNA).

Desirably, fragments from all the mentioned target genes are immobilized. In view of ensuring achievement of more efficient measurement of sensitivity, it is particularly preferred that fragments of at least 13 different genes in total be immobilized, including the following 11 genes; i.e., genes coding for TS, DPD, OPRT, TP, TK1, ERCC1, UDG, TOP1, MDR1, ENT1, and MRP1, as well as two or more of the genes coding for GAPDH, ACTB, or RSP9.

Regarding sequences of these target genes, a homology search is performed utilizing databases, whereby fragments which have high specificity to respective target genes are selected (Step 1).

The homology search may be carried out by use of, for example, Blast Search. Fragments may be designed as follows: GC content=40 to 60%; reduced number of duplicate sequences; Tm=75 to 85° C., size=200 to 600 bp.

RNA obtained from tumor cells are subjected to Northern hybridization, using the fragments designed in step 1, whereby specificity is confirmed (step 2). Theoretically, the fragments designed in step 1 have high specificity to target genes. However, in reality, cross-hybridization occurs at high probability. In fact, only 10 to 20% of the fragments selected in step 1 are confirmed to have specificity in step 2.

In step 2, firstly, PCR amplification is performed through use of a cDNA library as a template, whereby a DNA fragment is obtained. Next, while the thus-obtained DNA fragment is used as a template, radioactive probes are enzymatically synthesized, and by use of a membrane bearing blots of total RNA samples prepared from various tumor cells, Northern hybridization is performed, whereby specificity is checked. Briefly, presence of specificity is confirmed when a signal that corresponds to the size of mRNA transcribed from a certain gene is detected (the size information can be obtained from literature or database searches) and almost no other signals are detected (which means almost no cross-hybridization).

If a DNA fragment is found to have insufficient specificity, by use of another region of a corresponding gene another DNA fragment is designed, and then the re-designed fragment is subjected to Northern hybridization. This procedure is repeated until sufficient specificity is obtained. Thus, regions of the DNA fragments to be arrayed can be optimized. Each of the thus-obtained target fragments is cloned into a plasmid (pCR2.1-TOPO).

In this connection, as primers to be used in PCR amplification of target fragments, universal primers are designed on the basis of the sequence of multiple cloning site of a cloning vector, so that all the target fragments can be amplified by use of a set of universal primers. Especially preferred universal primers are those having the nucleotide sequences of SEQ ID NOs: 1 and 2. Also, in order to minimize the volume of primers that migrate into the spotting solution, the universal primer volume is optimized. When these measures are taken, background of every spot from the target fragments can be suppressed, with reduced differences.

When the thus-obtained target fragments of respective target genes are immobilized onto a substrate (support), the DNA array of the present invention can be obtained. The substrate used herein is any suitable one known per se. For example, mention may be given of glass plate, plastic plate, membrane (nylon membrane, etc.), and beads. Preferably, a glass plate is used. Immobilization of the fragments onto the array may be achieved by use of conventional spotting means. For example, there may be employed the surface adhesion method (the Stanford method) as described in PCT Kohyo Publication No. 10-503841, in which a tip-split pin is dipped into a solution containing the target DNA, and tapped onto a support for transfer; the ink-jet piezoelectric discharge method, in which a solution containing the target DNA is jetted onto a support, under the same principle that used in an ink jet printer; and the photolithograph methodology, which makes use of photolithography techniques and the target DNA is directly synthesized on a support. Preferably, the Stanford method is used.

Preferably, mRNA of all the target genes is quantitated, and this quantitation can be achieved by modifying the amount of a target fragment to be immobilized in accordance with each gene's expression level obtained through Northern hybridization; i.e., the amount of target fragments to be immobilized on the substrate (support) is reduced for a gene of higher expression level, and increased for a gene of lower expression level.

In order to measure the sensitivity to the aforementioned anticancer agents by use of the thus-obtained DNA array of the present invention, the DNA array is hybridized with labeled cDNA probes synthesized by using, as a template, mRNA obtained from a body fluid specimen or tissue specimen collected form a cancer patient.

Examples of the body fluid specimen originating from a cancer patient include blood and urine. Examples of the tissue include cancerous tissue. Collection of mRNA from a specimen is carried out through a conventional method, and mRNA may be in the form of “as contained” in total RNA, or may be isolated from the total RNA. To prepare the labeled cDNA, mRNA is used as a template, and reverse transcription enzyme reaction is performed for labeling. Examples of labeling means include fluorescent substances and radioisotopes, with fluorescent substances being especially preferred.

Hybridization may be carried out under conventional conditions. Quantitation of hybridization may be achieved through quantitating the amount of labeled probes; for example, intensity of fluorescence.

In order to validate the quantitativeness of the DNA array of the present invention, the measurements in terms of expression level of 52 species of genes were compared with the measurements obtained from Northern hybridization. As a result, almost no discrepancy was found. Discernment of a gene that shows a unique expression characteristic (i.e., a gene exhibiting an expression level twice or more the average expression level) was able to be obtained at the same precision as attained by Northern hybridization.

### EXAMPLES

The present invention will next be described in more detail by way of examples, which should not be construed as limiting the invention thereto.

### Example 1

(1) Preparation of Target Fragments

A. Selection and Design of DNA Fragments

As shown in Tables 2 and 3 below, 52 species of DNA fragments were selected as DNA fragments relating to 5-FU sensitivity, primarily from nucleic acid metabolism-related genes. In principle, the DNA fragments were designed such that each DNA fragment has a GC content of 40 to 60%, has almost no regions having the same nucleotide sequence as that of other DNA fragments of the same gene, has a Tm of 75 to 85° C., and has a size of 200 to 600 bp. In order to prevent cross-hybridization to the greatest possible extent, whether or not each of the DNA fragments had substantially no regions having the same nucleotide sequences as those of other DNA fragments in the same gene was investigated through a blast search by use of a database. By use of design-aiding software (Primer Express™, PE Biosystems), primers specific to each DNA fragment (specific primers [forward/reverse]), which were employed in PCR amplification, were designed so as to have a Tm of 59 to 61° C.

B. Synthesis and Purification of DNA Fragments

Each DNA fragment was amplified through PCR (30 cycles of treatment in total, each cycle consisting of thermal denaturation (94° C., 1 minute), annealing (60° C., 1 minute), and elongation (72° C., 1 minute)) while a human-derived cDNA library was used as a template, and by use of specific primers and an ExTaq™ (TaKaRa). The PCR product solution was subjected to purification through use of a spin column (Miniprep spin column, Aetna), and the purified product was eluted with distilled water.

(2) Northern Hybridization

A. Preparation and Purification of Total RNA Samples

A total RNA sample was extracted from each of the 14 types of human tumor cells listed in Table 1, through use of an RNeasy midi kit™ (QIAGEN) and eluted by distilled water. DNA fragments which might migrate in the solution were decomposed with DNase (37° C., 30 minutes), and proteins were removed by use of phenol/chloroform. The resultant solution was subjected to purification and condensation through ethanol precipitation, and the product was dissolved in distilled water, to thereby prepare a total RNA solution.

TABLE 1

| | Tumor type | Cell strain |
|---|---|---|
| 1 | Human gastric cancer | NUGC-3 |
| 2 | Human gastric cancer (FU-resistant) | NUGC-3/FU |
| 3 | Human large intestine cancer | DLD-1 |
| 4 | Human large intestine cancer (FU-resistant) | DLD-1/FU |
| 5 | Human large intestine cancer (FdUrd | DLD-1/FdUrd |
| | resistant) | |
| 6 | Human large intestine cancer (F3dThd- | DLD-1/F3dThd |
| | resistant) | |
| 7 | Human fibrosarcoma | HT1080 |
| 8 | Human fibrosarcoma (EUrd-resistant) | HT1080/EUrd |
| 9 | Human pancreatic carcinoma | MIAPaCa-2 |
| 10 | Human large intestine cancer | KM-12C |
| 11 | Human lung cancer | A549 |
| 12 | Human mammary cancer | MCF-7 |
| 13 | Human head and neck cancer | KB |
| 14 | Human ovarian cancer | TYK-nu |

Northern Blotting

The total RNA sample (5 μg) which had been prepared from each of the above 14 types of human tumor cells was electrophoresed on 1-wt % denatured agarose gel. The gel was stained with ethidium bromide, and the resultant gel was photographed under irradiation of UV rays, to thereby confirm that the RNA molecules had not been decomposed. The RNA molecules in the gel were blotted on a nylon membrane through a capillary phenomenon, and the membrane was subjected to fixation (cross-linking) under irradiation with UV rays.

C. Preparation of Probes

Probes labeled with [α-³²P]dCTP were synthesized by use of each of the DNA fragments prepared in (1) above as a template, through random priming (rediprime™ II, Amersham Pharmacia). [α-³²P]dCTP which had not been incorporated into the probes was removed through gel filtration (ProbeQuant™ G-50 micro spin column, Amersham Pharmacia).

D. Northern Hybridization

Northern hybridization was performed by use of the blot and the probes prepared in (2)B and (2)C above, respectively. Specifically, pre-hybridization was performed by use of the blot in a hybridization buffer (Rapid-hyb Buffer, Amersham Pharmacia) for 30 minutes at 65° C., the probes which had been undergone thermal denaturation were added thereto, and hybridization was caused to proceed for two hours at 65° C. Subsequently, the resultant blot was washed (twice with a 2×SSC solution (0.15-mol/L NaCl/0.15-mol/L trisodium citrate) containing 0.1-wt % SDS, once with a 1×SSC solution containing 0.1-wt % SDS, and twice with a 0.1×SSC solution containing 0.1-wt % SDS), and, in the dark, an imaging plate (Fujifilm) was exposed with the blot overnight. On the following day, the imaging plate was scanned by means of an imaging and analysis apparatus (STORM, Molecular Dynamics Inc.), and the obtained image data were stored.

E. Evaluation and Redesign of DNA Fragments

Specificity of DNA fragments was evaluated from the images obtained through Northern hybridization. Specifically, when a signal was observed at a position corresponding to the size of mRNA of the gene of interest, and substantially no other signals were detected, the DNA fragment was determined to have specificity, whereas when no signal was observed at a position corresponding to the size of mRNA of the gene of interest, or when other signals were detected (cross-hybridization), the DNA fragment was determined to have poor specificity. In the latter case, another DNA fragment that would match the condition described in (1)A above was engineered in a different region of mRNA of the same gene. Subsequently, until such a redesigned DNA fragment was confirmed to have specificity, a cycle consisting of design, synthesis, and Northern hybridization was repeated (in some cases, the cycle was repeated six times), whereby the target fragment, the region of the gene to be fixed onto the DNA array of the present invention, was determined.

(3) Preparation of DNA Array and Hybridization

The following steps are based on the Stanford method, which was partially modified.

A. Cloning of Target Fragments

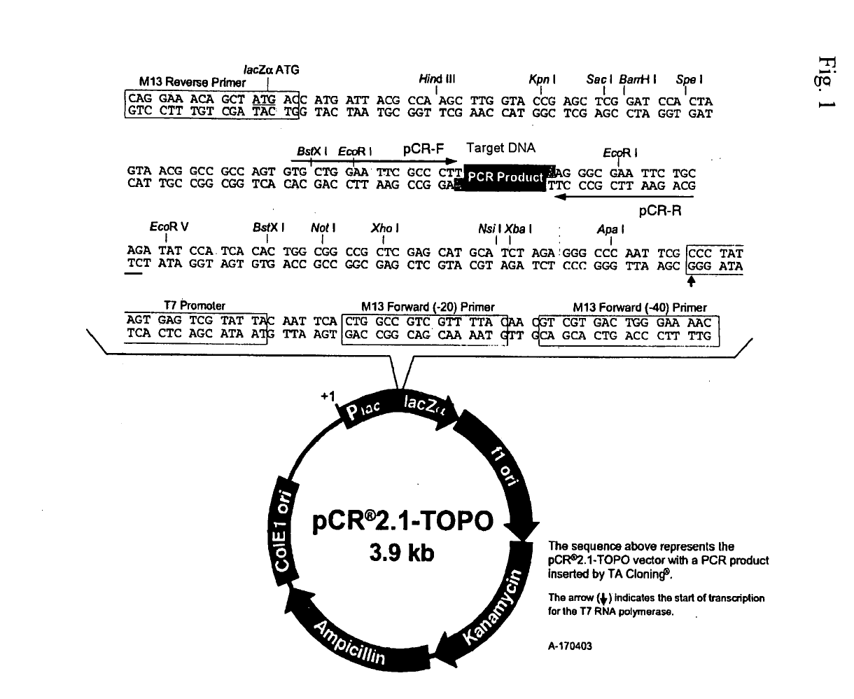

For preparing sufficient amounts of target fragments having good quality, use of cDNA library as a template is not preferred, since quality of cDNA library differs from lot to lot. Therefore, as shown in FIG. 1, each of the target fragments (the PCR products) was subjected to cloning through TA cloning by use of a plasmid (pCR-TOPO vector, Invitrogen), to thereby prepare a clone corresponding to the target fragment (52 species in total).

B. Preparation of Target Fragments

The target fragment was amplified in accordance with the method described in (1) above through use of the clone as a template. The employed primers were universal primers (SEQ ID NOS: 1 and 2). The PCR product was subjected to ethanol precipitation, and the collected precipitates were washed with ethanol and dissolved in distilled water. An aliquot of the solution was employed for calculation of concentration (through absorptiometry) and assay for determining purification (through electrophoresis on agarose gel). The remaining target fragment solution was dried at room temperature under reduced pressure, and then the fragment was dissolved in Micro Spotting Solution (BM) so as to have a concentration of 0.5 to 10 pmol/μL (Tables 2 and 3 show the concentration of each target fragment). Nucleotide sequences of the thus-obtained target fragments are shown by SEQ ID NOs: 3 to 54.

TABLE 2

| | | | Amount of DNA | | |
|---|---|---|---|---|---|
| | | GenBank | on tip | Region [nt] (length [bp]) | Number of Northern |
| Name of gene | Abbreviation | Accession# | (pmol/μL) | of target fragment | hybridization performed |
| Nucleic acid metabolism-related enzyme | | | | | |
| Thymidylate synthase | TS | NM_001071 | 1 | 1099-1466 (368) | 1 |
| Dihydropyrimidine dehydrogenase | DPD | U09178 | 10 | 1150-1537 (388) | 4 |
| Orotate phosphoribosyltransferase | OPRT | NM_000373 | 2 | 1299-1662 (364) | 1 |
| (uridine monophosphate synthetase) | (UMPS) | | | | |
| Thymidine phosphorylase | TP | M63193 | 10 | 490-797 (308) | 4 |
| Thymidine kinase 1 | TK1 | NM_003258 | 2.5 | 595-908 (314) | 2 |
| Ribonucleoside-diphosphate reductase M1 subunit | RRM1 | X59543 | 5 | 1669-2010 (342) | 3 |
| Ribonucleoside-diphosphate reductase M2 subunit | RRM2 | NM_001034 | 1 | 1840-2097 (258) | 3 |
| Uridine cytidine kinase 2 | UCK2 | AF236637 | 2.5 | 61-609 (549) | 3 |
| Uridine phosphorylase | UP | NM_003364 | 10 | 931-1314 (384) | 3 |
| Cytidine deaminase | CDA | L27943 | 5 | 304-605 (302) | 3 |
| 5′ nucleotidase | NT5 | NM_002526 | 5 | 2800-3073 (274) | 3 |
| IMP dehydrogenase 1 | IMPD | NM_000883 | 5 | 1967-2220 (254) | 4 |
| Methylenetetrahydrofolate dehydrogenase | MTHFD1 | NM_005956 | 2.5 | 1768-2071 (304) | 3 |
| RNA polymerase 2 | RP2 | NM_000937 | 2.5 | 1457-1787 (331) | 6 |
| Uridine monophosphate kinase | UMPK | NM_016308 | 5 | 719-1003 (285) | 4 |
| CTP synthase | CTPS | NM_001905 | 2.5 | 2130-2394 (265) | 3 |
| Deoxycytidylate deaminase | DCD | NM_001921 | 5 | 1176-1543 (368) | 3 |
| Deoxycytidine kinase | DCK | NM_000788 | 5 | 542-963 (422) | 2 |
| Phosphoribosyl pyrophosphate synthetase | PRPS | D00860 | 5 | 1152-1473 (322) | 3 |
| Hypoxanthine phosphoribosyltransferase 1 | HPRT1 | NM_000194 | 2.5 | 824-1214 (391) | 2 |
| Folylpolyglutamate synthetase | FPGS | NM_004957 | 10 | 92-311 (220) | 3 |
| Nucleoside diphosphate kinase A | NDKA | X17620 | 0.5 | 299-662 (364) | 1 |
| Nucleoside diphosphate kinase B | NDKB | L16785 | 0.5 | 210-581 (372) | 1 |
| Adenine phosphoribosyltransferase | APRT | NM_000485 | 5 | 154-559 (406) | 1 |
| Adenosine kinase | AK | U33936 | 5 | 651-1019 (369) | 1 |

TABLE 3

| | | | Amount of DNA | | |
|---|---|---|---|---|---|
| | | GenBank | on tip | Region [nt] (length [bp]) | Number of Northern |
| Name of gene | Abbreviation | Accession# | (pmol/μL) | of target fragment | hybridization performed |
| Gene repair-associated enzyme | | | | | |
| DNA excision repair protein ERCC1 | ERCC1 | NM_001983 | 5 | 210-526 (317) | 2 |
| Uracil-DNA glycosylase | UDG | X15653 | 2.5 | 1553-1943 (391) | 1 |
| Poly(ADP-ribose)polymerase | PARP | NM_001618 | 2.5 | 2684-3093 (410) | 3 |
| DNA ligase I | LIG1 | NM_000234 | 5 | 1363-1671 (309) | 3 |
| DNA ligase III | LIG3 | X84740 | 10 | 1680-2034 (355) | 2 |
| DNA ligase IV | LIG4 | NM_002312 | 10 | 2088-2498 (411) | 4 |
| DNA polymerase β | POLB | NM_002690 | 5 | 110-444 (335) | 2 |
| DNA polymerase δ | POLD | NM_002691 | 5 | 1198-1459 (262) | 3 |
| DNA-repair protein XRCC1 | XRCC1 | NM_006297 | 10 | 898-1265 (368) | 3 |
| Drug resistance-related factor | | | | | |
| Topoisomerase 1 | TOP1 | J03250 | 2.5 | 1133-1456 (324) | 3 |
| P-glycoprotein | MDR1 | NM_000927 | 5 | 1617-1881 (265) | 3 |
| Equilibrative nucleoside transporter 1 | ENT1 | NM_004955 | 2.5 | 148-403 (256) | 3 |
| Multidrug resistance-associated | MRP1 | L05628 | 10 | 3841-4239 (399) | 5 |
| protein 1 | | | | | |
| Topoisomerase 2 α | TOP2A | NM_001067 | 2.5 | 3037-3389 (353) | 6 |
| Topoisomerase 2 β | TOP2B | X68060 | 2.5 | 3142-3515 (374) | 3 |
| Heat shock protein 27 | Hsp27 | NM_001540 | 1 | 323-534 (212) | 3 |
| Equilibrative nucleoside transporter 2 | ENT2 | AF034102 | 5 | 1941-2263 (323) | 3 |
| Others | | | | | |
| E2F1 | E2F1 | M96577 | 5 | 1014-1309 (296) | 5 |
| p53 | p53 | NM_000546 | 2.5 | 796-1130 (335) | 2 |
| VEGF β | VEGFB | U48801 | 10 | 97-375 (279) | 1 |
| Integrin α 3 | ITGA3 | NM_002204 | 2.5 | 2563-2930 (368) | 3 |
| Mn SOD | SOD2 | NM_000636 | 2.5 | 60-329 (270) | 1 |
| Cu/Zn SOD | SOD1 | X02317 | 2.5 | 53-312 (260) | 2 |
| Proliferating cell nuclear antigen | PCNA | NM_002592 | 1 | 108-423 (316) | 1 |
| Housekeeping gene | | | | | |
| (internal standard) | | | | | |
| Glyceraldehyde-3-phosphate | GAPDH | X01677 | 0.5 | 453-785 (333) | 2 |
| dehydrogenase | | | | | |
| β-actin | ACTB | NM_001101 | 0.5 | 820-1083 (264) | 2 |
| 40S ribosomal protein S9 | RSP9 | U14971 | 0.5 | 325-685 (361) | 1 |

C. Spotting and Post-Treatment

The target fragment which had undergone thermal denaturation for three minutes at 95° C. was spotted onto a glass slide coated with poly-L-lysine, by means of a spotter (OmniGrid, GENEMACHINES). Subsequently, the target fragment was cross-linked with the glass slide under irradiation with UV rays, and the glass slide was placed in a rack and shaken in a blocking solution (8-wt % Block A in PBS) for 30 minutes, and the resultant slide was washed with a TE buffer and dried. The slide glass was stored in a desiccator in the dark until the time of use. Following the process, the DNA array of the present invention was obtained.

D. Preparation of Fluorescence-Labeled DNA Probes

Reverse transcription reaction was performed through use of a total RNA sample prepared from tumor cells (through the same method as described in (2) above) as a template and primers specific to mRNA of each gene, to thereby prepare fluorescence-labeled probes. Reagents employed for labeling reaction are as follows.

reverse transcriptase SuperscriptII (Gibco BRL)

(reaction buffer, DTT)

a primer mixture (a mixture of reverse primers specific to each of the 52 genes; the reverse primers are one of the specific primers employed in Referential Example 1)

dATP, dGTP, dCTP, dTTP (Amersham Pharmacia)

Cy3-dUTP (Amersham Pharmacia)

Cy5-dUTP (Amersham Pharmacia)

0.5M EDTA

1N NaOH

1M Tris-HCl (pH 7.5)

TE buffer

Specifically, a total RNA sample (30 μg) was mixed with a primer mixture (50 pmol each) and distilled water, and the volume of the resultant mixture was adjusted to 9 μL. The mixture was denatured for two minutes at 65° C. and rapidly cooled on ice. A reaction buffer (1×), DTT (10 mM), dTTP (0.2 mM), DATP (0.5 mM), dGTP (0.5 mM), dCTP (0.5 mM), Cy3-dUTP or Cy5-dUTP (0.1 mM), and Superscript II (10 U/μL) were added thereto, and the total volume of the mixture was adjusted with distilled water to 20 μL (the concentrations in the parentheses refer to final concentrations). The mixture was allowed to react for 60 minutes at 42° C., and distilled water (20 μL), 0.5M EDTA (5 μL), and 1N NaOH (5 μL) were added thereto. The resultant mixture was incubated for 60 minutes at 65° C., whereby total RNA molecules were decomposed. The reaction mixture was neutralized with 1M Tris-HCl (25 μL). A TE buffer (200 to 400 μL) was added thereto, and the resultant mixture was desalted and concentrated through ultrafiltration (Microcon-30, Millipore) (in this step, reverse primers and Cy3-dUTP or Cy5-dUTP which had not been incorporated into probes were also removed). Ultimately, about 10 μL of a probe solution was obtained.

E. Hybridization

20×SSC (3 μL) and distilled water were added to the above fluorescence-labeled DNA probe solution, and the volume of the mixture was adjusted to 20 μL (final concentration: 3×SSC). The mixture was subjected to thermal denaturation for three minutes at 95° C., and the reaction mixture was left to stand at room temperature for cooling. 10-wt % SDS (2 μL) and distilled water were added to the resultant mixture, and the volume of the mixture was adjusted to 40 μL (final concentration: 0.5 wt %). The mixture was added dropwise to the DNA array prepared in the steps A to C, and a glass cover was gently placed onto the array (so as not to allow air inside). The DNA array was set in a hybridization chamber and incubated for 10 to 20 hours at 65° C. Thereafter, the DNA array was placed in a 2×SSC solution containing 0.2-wt % SDS, and the glass cover was gently removed. The DNA array was washed in situ for five minutes. Moreover, the DNA array was washed once with a 2×SSC solution containing 0.2-wt % SDS and twice with a 0.2×SSC solution containing 0.2-wt % SDS, and rinsed twice with 0.2×SSC (all of these washing steps were performed at room temperature). The DNA array was placed in a rack and centrifuged for 20 seconds at 600 rpm, to thereby remove water. The DNA array was dried at room temperature, and fluorescent signals from the DNA array were measured by means of a DNA microarray fluorescence scanner (GenePix, Axon).

Test 1: Specificity of DNA Fragments

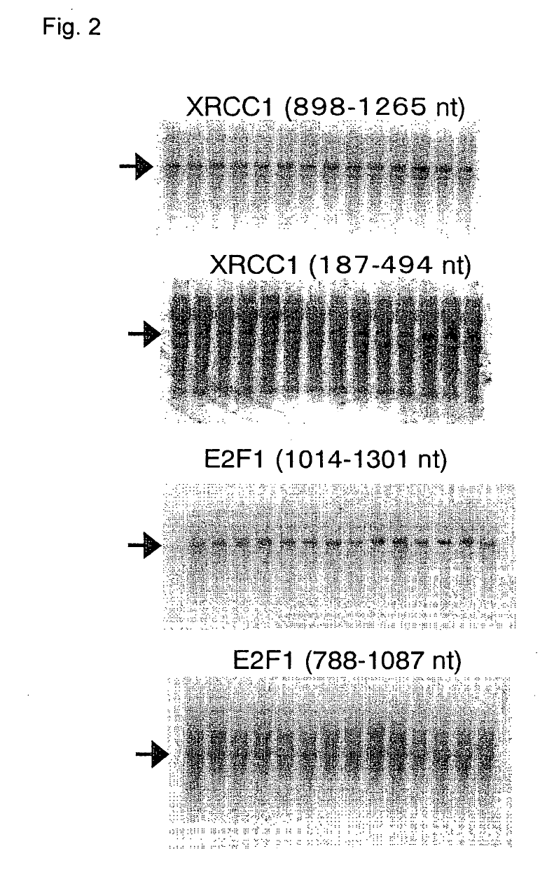

Specificity of DNA fragments employed as templates for synthesis of probes in Northern hybridization differed from fragment to fragment, although the fragments were contained in the same gene. FIG. 2 shows some of the test results, using XRCC1 and E2F1. In XRCC1, a region of 898 to 1265 nt exhibited good results (strong signals were detected at a position corresponding to the size of mRNA of the gene of interest), and a region of 187 to 494 nt was determined not to be suitable, since a large number of signals indicating cross-hybridization were observed. Similarly, in E2F1, a region of 1014 to 1309 nt exhibited good results, and a region of 788 to 1087 nt was determined not to be suitable. When regions of other genes were investigated, various levels of specificity were also observed, although the levels differed from gene to gene. A DNA fragment which exhibited the highest specificity in DNA fragments of gene of interest was selected in the final step and was employed as a target fragment.

Test 2: Performance of Northern Hybridization in Terms of Quantitativeness

Performance of DNA arrays in terms of quantitativeness is confirmed by comparison with Northern hybridization. Accordingly, in order to confirm the performance of the measurement system of Northern hybridization employed by the present inventors (Example 1(2)), correlation of the RNA level and measurements (signal intensity) was investigated.

A total RNA sample derived from each of the above 14 types of human tumor cells was electrophoresed on a denatured agarose gel and blotted on a nylon membrane. Northern hybridization was performed through use of probes synthesized from target DNA of GAPDH (glyceraldehydes-3-phosphate dehydrogenase) (through the method described in Example 1(2)). Analysis was performed by means of imaging and analysis software (ImageQuant, Molecular Dynamics Inc), and intensity of GAPDH mRNA signals was measured.

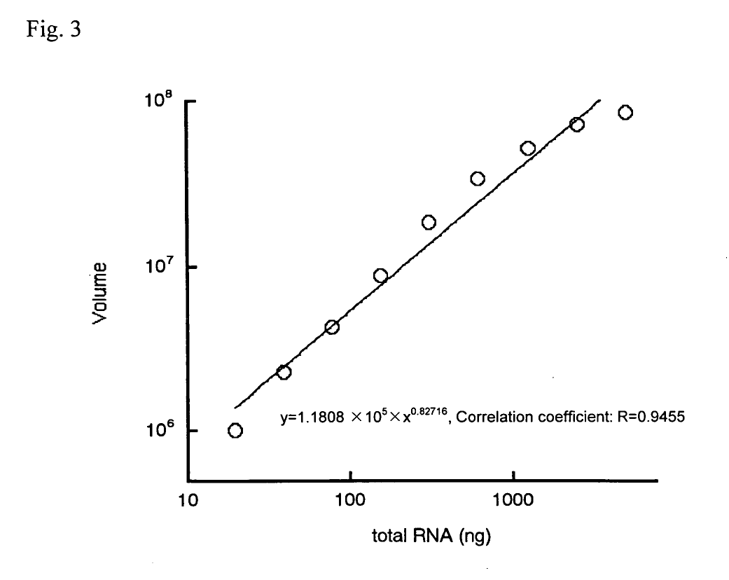

FIG. 3 shows the relationship between the amount of the electrophoresed total RNA sample and measurements of Northern hybridization. The results confirmed that a linear relationship exists between the amount of the total RNA sample and signal intensity (r=0.95, p<0.01), indicating that the measurement system employed provides high performance in terms of quantitativeness.

Test 3: Effect of Universal Primers on Background

As shown in FIG. 1, universal primers (forward (pCR-F)/reverse (pCR-R)) were designed on the basis of the sequence of a multicloning site of vector. Since all 52 target fragments can be amplified at a constant rate by using universal primers, target fragments can be readily provided in sufficient amounts as compared with the case of specific primers.

In order to determine the effect of primers which may migrate into target fragments upon spotting on measurement background, universal primers (5 spots) and specific primers (primers corresponding to each of about 20-bp sequences at both ends of 5 target DNA fragments [TS, DPD, OPRT, LIG4, and GAPDH] listed in Tables 2 and 3, 1 spot each) were spotted in equal amounts onto glass slides. The glass slides were subjected to the hybridization procedure described in Example 1(3), and signal intensity was measured.

The mean value, standard deviation (SD), and coefficient of variation (CV) of signal intensities obtained from the universal primers (5 spots) were found to be 27.3, 6.5, and 24.0%, respectively. The mean value, standard deviation (SD), and coefficient of variation (CV) of signal intensities obtained from the specific primers (5 spots in total) were found to be 22.9, 12.0, and 52.4%, respectively. Signal intensities from both primers were found to be of substantially the same level, and signal intensities obtained from the universal primers had lower SD and CV, indicating that the universal primers exert less effect on difference in background level than the specific primers.

In addition, in order to reduce background to the lowest level as possible, the minimum amount of the universal primers required to amplify a target fragment was studied. PCR was performed through use of forward and reverse primers. The amount of each primer was 10, 20, 30, 40, and 50 pmol. The PCR products were electrophoresed on agarose gel. When primers were used in amounts of 20 pmol or less, the amount of PCR product was found to be reduced. Therefore, the minimum amount of the universal primers required to amplify a target fragment was determined to be 30 pmol.

### Example 2

### Measurement of Expression Level of Various Genes in Human Tumor Cells

(1) Measurement Through Northern Hybridization

In the step of determining target fragments to be placed onto the DNA array, the expression level of each of the 52 genes was determined through analysis of blot images obtained through Northern hybridization of the above 14 different human tumor cells (see Example 1(2)). The expression level is represented by the value relative to the mean value of the expression levels of three housekeeping genes (GAPDH, ACTB, and RSP9).

(2) Measurement by Use of DNA Array

A DNA array onto which the 52 DNA target fragments were immobilized was prepared, and hybridization was performed through use of fluorescent DNA probes prepared from the total RNA samples which had been extracted from the 14 types of cells and employed in Northern hybridization. The expression level of each gene was measured (through the same method as described in Example 1 (3)). The expression level is represented by the value relative to the mean value of the expression levels of three housekeeping genes (GAPDH, ACTB, and RSP9).

(3) Correlation of the Results Obtained Through Northern Hybridization with the Results Obtained Through DNA Array

In order to determine whether the expression levels of each of the 52 species of genes determined through Northern hybridization in the above 14 species of human tumor cells were correlated with those determined by use of DNA arrays, regression analysis was performed (Table 4). The degree of correlation (correlation coefficient) differed from gene species to gene species, and significant correlation was confirmed in 20 species of genes at a significance level of less than 5%. Some degree of correlation was confirmed in nine species of genes, although the degree was not significant (p<0.1). Twelve species of genes containing three species of housekeeping genes (MTHFD1, SOD1, AK, E2F1, POLD, LIG3, RSP9, HPRT1, ACTB, UDG, GAPDH, and LIG1) were found to have only a low degree of correlation or were found impossible to evaluate, since difference in expression level between cells were small; in other words, the normal distribution was not obtained.

TABLE 4

| | R | | | |
|---|---|---|---|---|
| | (correlation | | | Significance |
| Abbreviation | coefficient) | P-value | Correlation | (P < 0.05) |
| ERCC1 | 0.90 | 9.4E−06 | High | Yes |
| p53 | 0.90 | 1.1E−05 | High | Yes |
| MDR1 | 0.90 | 1.3E−05 | High | Yes |
| DPD | 0.89 | 2.2E−05 | High | Yes |
| CDA | 0.87 | 5.0E−05 | High | Yes |
| TS | 0.85 | 1.1E−04 | High | Yes |
| PRPS | 0.85 | 1.2E−04 | High | Yes |
| NDKB | 0.80 | 6.5E−04 | Relatively high | Yes |
| POLB | 0.79 | 7.3E−04 | Relatively high | Yes |
| XRCC1 | 0.77 | 1.3E−03 | Relatively high | Yes |
| MRP1 | 0.76 | 1.4E−03 | Relatively high | Yes |
| ENT2 | 0.75 | 2.0E−03 | Relatively high | Yes |
| APRT | 0.75 | 2.2E−03 | Relatively high | Yes |
| Hsp27 | 0.67 | 8.2E−03 | Relatively high | Yes |
| VEGFB | 0.60 | 2.2E−02 | Relatively high | Yes |
| TOP2B | 0.57 | 3.3E−02 | Medium | Yes |
| LIG4 | 0.57 | 3.4E−02 | Medium | Yes |
| ENT1 | 0.55 | 4.3E−02 | Medium | Yes |
| RRM2 | 0.54 | 4.6E−02 | Medium | Yes |
| IMPD | 0.54 | 4.8E−02 | Medium | Yes |
| NDKA | 0.53 | 5.3E−02 | Medium | No |
| RP2 | 0.51 | 6.2E−02 | Medium | No |
| TP | 0.50 | 7.1E−02 | Medium | No |
| RRM1 | 0.49 | 7.7E−02 | Medium | No |
| UCK2 | 0.45 | 1.1E−01 | Medium | No |
| FPGS | 0.44 | 1.2E−01 | Medium | No |
| CTPS | 0.44 | 1.2E−01 | Medium | No |
| UMPS(OPRT) | 0.42 | 1.3E−01 | Medium | No |
| SOD2 | 0.42 | 1.4E−01 | Medium | No |
| UP | 0.39 | 1.6E−01 | Relatively low | No |
| NT5 | 0.34 | 2.3E−01 | Relatively low | No |
| TK1 | 0.34 | 2.4E−01 | Relatively low | No |
| DCK | 0.33 | 2.5E−01 | Relatively low | No |
| TOP2A | 0.32 | 2.7E−01 | Relatively low | No |
| DCD | 0.27 | 3.5E−01 | Relatively low | No |
| ITGA3 | 0.26 | 3.7E−01 | Relatively low | No |
| PARP | 0.24 | 4.1E−01 | Relatively low | No |
| TOP1 | 0.23 | 4.3E−01 | Relatively low | No |
| PCNA | 0.23 | 4.3E−01 | Relatively low | No |
| UMPK | 0.21 | 4.6E−01 | Relatively low | No |
| LIG1 | 0.06 | 8.3E−01 | Low | No |
| MTHFD1 | 0.40 | 1.6E−01 | Impossible | No |
| | | | to evaluate | |
| SOD1 | 0.39 | 1.6E−01 | Impossible | No |
| | | | to evaluate | |
| AK | 0.37 | 1.9E−01 | Impossible | No |
| | | | to evaluate | |
| E2F1 | 0.29 | 3.2E−01 | Impossible | No |
| | | | to evaluate | |
| POLD | 0.28 | 3.3E−01 | Impossible | No |
| | | | to evaluate | |
| LIG3 | 0.27 | 3.4E−01 | Impossible | No |
| | | | to evaluate | |
| RSP9 | 0.26 | 3.7E−01 | Impossible | No |
| | | | to evaluate | |
| HPRT1 | 0.25 | 3.8E−01 | Impossible | No |
| | | | to evaluate | |
| ACTB | 0.20 | 5.0E−01 | Impossible | No |
| | | | to evaluate | |
| UDG | 0.17 | 5.7E−01 | Impossible | No |
| | | | to evaluate | |
| GAPDH | 0.14 | 6.4E−01 | Impossible | No |
| | | | to evaluate | |

(4) Performance of DNA Array in Terms of Judgment

When such a tool used for the purpose of diagnosis (proper use of a drug) is employed in actual clinical sites, whether a sample is positive or negative is generally determined on the basis of threshold. For example, in research in which TS or DPD mRNA level determined through RT-PCR is used as an index which indicates proper use of 5-FU, a threshold has been set for determining that a level is high or low (Clinical Cancer Research, 6, 1322-1327, 2000). The performance of the DNA array of the present invention was determined through comparison with Northern hybridization in the following manner.

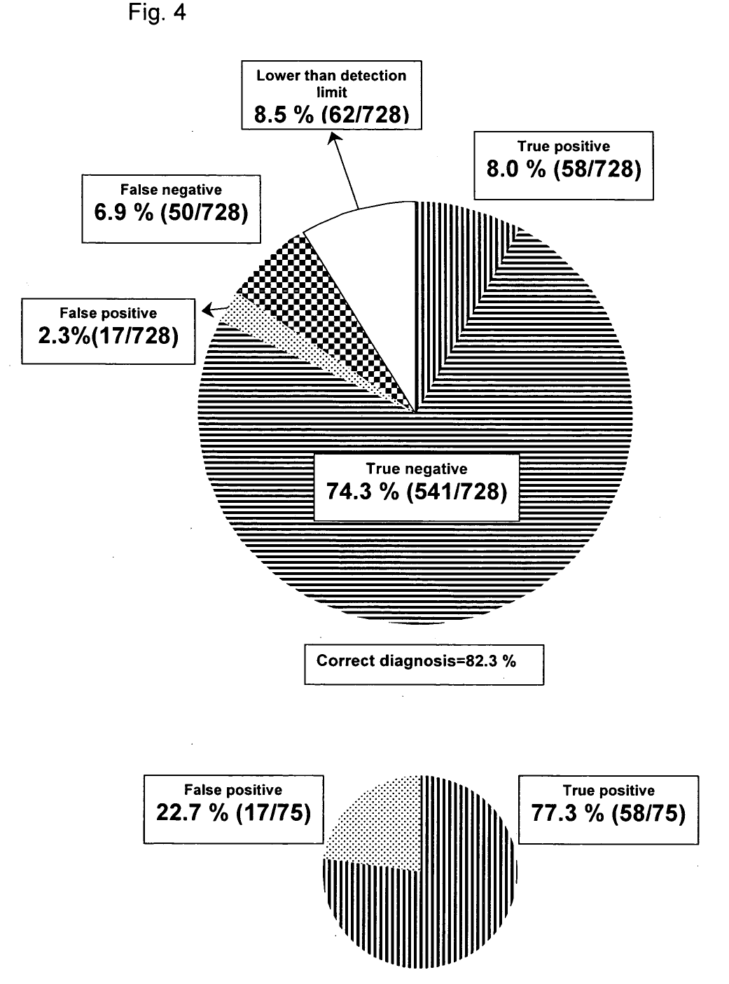

The above 14 species of human tumor cells were treated as clinical specimens. The expression level of each of the 52 species of genes in each type of cells was measured through use of the DNA array of the present invention or through Northern hybridization (in each method, 52 (genes)×14 (cells)=728 points were measured and calculated). From measurements obtained through use of the DNA array of the present invention, the median value of the expression levels of each gene in the 14 species of cells was calculated. The relative expression level (the ratio of expression level of each of the 14 species of cells to the median value (base)) was calculated. The threshold was set as 2-fold. When the relative expression level went beyond the threshold value (≧2 or ≦0.5), the cell was determined to be “positive,” whereas when the relative expression level did not exceed the threshold value, the cell was determined to be “negative.” On the assumption that results determined through Northern hybridization were highly reliable, when results determined by use of the DNA array of the present invention were identical with those determined through Northern hybridization, the results were evaluated as “true,” whereas when results determined by use of the DNA array of the present invention were different from those determined through Northern hybridization, the results were evaluated as “faulty.” As shown in FIG. 4, in the positive and negative results, the percentage of data evaluated “true” was found to be as high as 82.3%. In 75 points which had been determined as positive through use of the DNA array of the present invention, 77.3% (58/75) of the points presented the same results as those of Northern hybridization. In addition, there were no cases in which the measurements and results of the DNA array were inconsistent with those of Northern hybridization (for example, there were no cases in which both results were true positive but the relative expression level through Northern hybridization (e.g., 0.3) was quite different from those obtained through use of the DNA array of the present invention (e.g., 2.5)). Thus, the DNA array of the present invention was found to present substantially the same level of performance as that of Northern hybridization.

(5) Detecting Ability of DNA Array

In Test 4 (4), among the tested 728 points (52 (genes)×14 (cells)), 62 points were found to be impossible to measure. None of the 62 points could not be detected through Northern hybridization. Therefore, the results indicate that, as compared with Northern hybridization, the DNA array of the present invention presents the same level or a higher level of detecting ability.

### Example 3

### Correlation between TS-1 Sensitivity and Expression Level of Various Genes in Transplantable Human Tumor Cells

(1) TS-1 Sensitivity Test

The DNA array of the present invention may be clinically applied to, for example, determining the genes that are important as sensitivity regulating factors, by analyzing correlation between expression levels of the 52 genes in cancer tissue and antitumor effect of a 5-FU anticancer agent.

In this test, as a test model for determining anticancer effect, TS-1 sensitivity of 11 species of transplantable human tumor cells (xenografts, derived from gastric cancer (4 types), large intestine cancer (3 types), lung cancer (2 types), and mammary cancer (2 types)) was tested (TS-1, a 5-FU anticancer agent which has been developed by Taiho Pharmaceutical Co., Ltd., is a composition-containing agent containing tegafur (5-FU prodrug), gimeracil (a DPD inhibitor), and oteracil potassium (an orotate phosphoribosyl transferase inhibitor) at a ratio by mole 1:0.4:1). Each xenograft was subcutaneously transplanted into nude mice at the back thereof. When the transplanted site was expanded to 100 to 200 mm³, the mice were divided into two groups; i.e., a control group (a group to which no drug was administered) and a TS-1 administered group (6 animals/group). From the following day, TS-1 was perorally administered to mice of the TS-1 administered group once a day for 14 successive days at a dose of 10 mg/kg/day (FT). On the day following the final administration, the volumes of the tumors were measured, and the volumes of the TS-1 administered group were compared with those of the control group. Tumor multiplication inhibition rate (IR) was determined. IR was calculated by the following equation.

IR(%)=(1-[Relative tumor volume of the TS-1 administered group]/[Relative tumor volume of the control group])×100

(Relative tumor volume)=(tumor volume upon judgement)/(tumor volume upon grouping)

(2) Measurement by Use of the DNA Array of the Present Invention

The expression levels of the 52 species of genes were determined through use of total RNA samples prepared from the 11 species of xenografts. The test was performed in accordance with the method described in Example 2. The expression level is represented by the value relative to the mean value of the expression levels of three species of housekeeping genes (GAPDH, ACTB, and RSP9).

(3) Correlation between the Expression Level in 11 Species of Xenografts and TS-1 Sensitivity

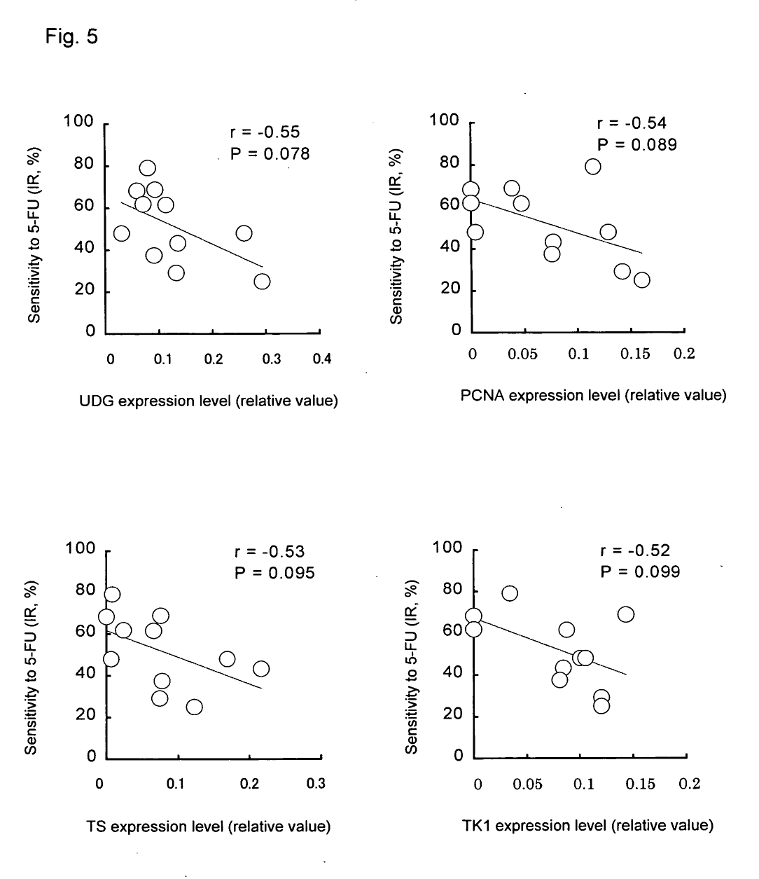

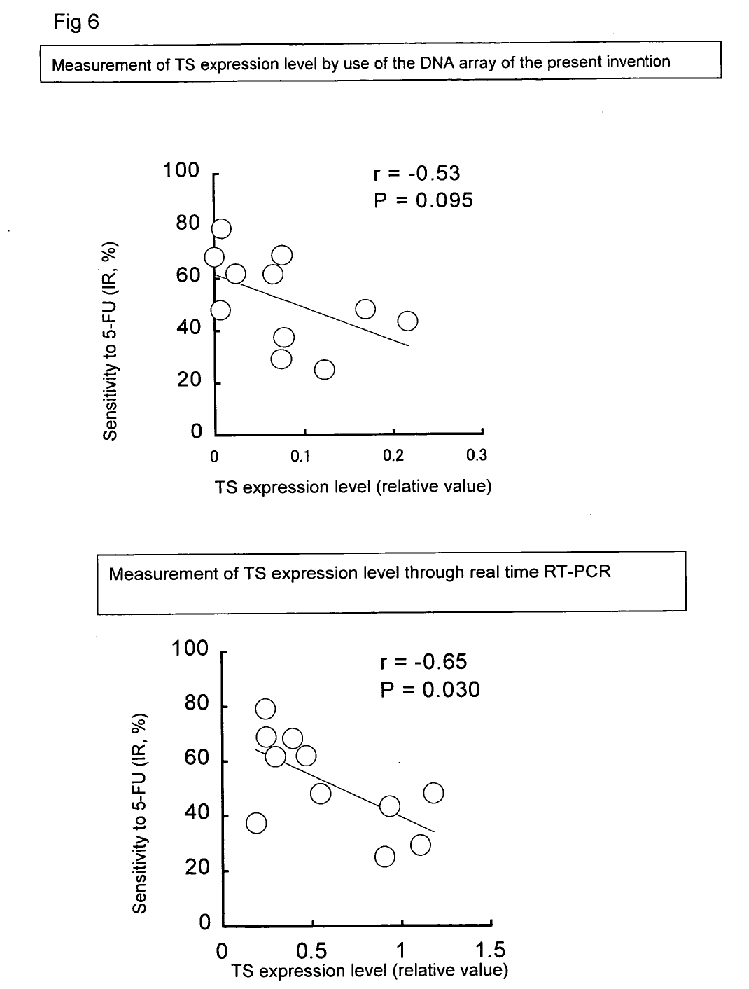

In order to determine whether the expression level of the 52 species of the genes in the 11 species of xenograft, determined in the studies (1) and (2) above, is correlated with TS-1 sensitivity (IR, %), regression analysis was performed (52 (genes)×11 (cells)). As a result, four species of the gene (UDG, PCNA, TS, and TK1) were found to have an absolute value of correlation coefficient (P-value: Pearson's product-moment correlation coefficient) higher than 0.5 (FIG. 5). P-value was found to be 0.05 or higher and lower than 0.1. Thus, the data were not statistically significant. However, since N is as small as 11, from the regression line, the expression level is confirmed to be substantially correlated with the sensitivity. It should be noted that these four species of genes contain TS. TS is a representative sensitivity regulating factor for 5-FU anticancer agent, and both basic research and clinical research have revealed that the higher the TS expression level, the lower the effect exhibited by a 5-FU anticancer agent. In the study of the present invention, TS expression level was found to have a negative correlation with antitumor effect of TS-1 (r=−0.53). The results coincide with the previous finding. In addition, as a confirmation test, the TS expression level in the 11 species of cells (relative expression level against GAPDH) was measured through real time RT-PCR, which is considered to provide considerably high measurement accuracy, to thereby analyze correlation between TS-1 sensitivity and the TS expression level. The correlation coefficient was found to be −0.65; i.e., a significant correlation (P=0.030) was confirmed (FIG. 6). These data indicate that the DNA array of the present invention is useful for determining candidates of sensitivity regulating factors of a 5-FU anticancer agent from the 52 species of gene. When N increases, clinical specimens may be used to determine sensitivity regulating factors of a 5-FU anticancer agent, and when the expression level of a selected gene was determined through analysis, the expression level may be used as an index which indicates proper use of 5-FU anticancer agent.

### INDUSTRIAL APPLICABILITY

The present invention achieves convenient and highly quantitative measurement of expression levels of several tens to several hundreds of gene species contained in a specimen in a single measurement. When expression patterns of genes related to action mechanisms of an antimetabolite-type anticancer agent or a combination of such an agent and another anticancer agent are analyzed through use of the assay method of the present invention in a test specimen (for example, total RNA extracted from peripheral monocytes or tumor tissue of a cancer patient), results obtained from such an analysis can be employed as indices for proper use of the anticancer agent.

1. A DNA array for measuring sensitivity to an antimetabolite-type anticancer agent or to a combination of such an anticancer agent and another anticancer agent, characterized by comprising a substrate and target gene fragments attached to the substrate,
the target gene fragments being of at least 13 gene species, including at least 2 species from each of the following gene groups: a group of genes coding for nucleic -acid-metabolism-related enzymes, a group of genes coding for gene-repair-associated enzymes, a group of genes coding for drug resistance-associated factors, and housekeeping genes, and said at least 13 gene species being selected by performing the following two steps 1) and 2):
1) selecting fragments having high target-gene specificity through a homology search using a database, and
2) performing Northern hybridization to RNA collected from tumor cells, using the fragments selected in step 1) as probes, to thereby confirm target-gene specificity.

2. The DNA array according to claim 1, wherein the amounts of respective fragments attached to the substrate are regulated in accordance with expression levels of the target genes in the tumor cells.

3. The DNA array according to claim 1 or 2, wherein the fragments selected in the step 2) are obtained through PCR employing universal primers having nucleotide sequences of SEQ ID NOs: 1 and 2.

4. The DNA array according to any one of claims 1 to 3, wherein:
the group of genes coding for nucleic-acid-metabolism-related enzymes includes at least thymidylate synthase gene, dihydropyrimidine dehydrogenase gene, orotate phosphoribosyltransferase gene, thymidine phosphorylase gene, and thymidine kinase 1 gene;
the group of genes coding for gene-repair-associated enzymes includes at least DNA excision repair protein ERCC1 gene and uracil-DNA glycosylase gene;
the group of genes coding for drug resistance-related factors include topoisomerase 1 gene, P-glycoprotein gene, equilibrative nucleoside transporter 1 gene, and multidrug resistance-associated protein 1 gene; and
the group of housekeeping genes consists of two or more genes selected from among glyceraldehyde-3-phosphate dehydrogenase gene, β-actin gene, and 40S ribosomal protein S9 gene.

5. The DNA array according to any one of claims 1 to 4, wherein:
each of the target gene fragments has a size of 200 to 600 bp.

6. A DNA array for measuring sensitivity to an antimetabolite-type anticancer agent or to a combination of such an anticancer agent and another anticancer agent, characterized by comprising a substrate and at least the following gene fragments (A), (B), (C), and (D) attached to the substrate:
(A) gene fragments selected from a nucleic-acid -metabolism-related enzyme gene group comprising a thymidylate synthase gene fragment having a nucleotide sequence of SEQ ID NO: 3, a dihydropyrimidine dehydrogenase gene fragment having a nucleotide sequence of SEQ ID NO: 4, an orotate phosphoribosyltransferase gene fragment having a nucleotide sequence of SEQ ID NO: 5, a thymidine phosphorylase gene fragment having a nucleotide sequence of SEQ ID NO: 6, and a thymidine kinase 1 gene fragment having a nucleotide sequence of SEQ ID NO: 7;
(B) gene fragments selected from a gene-repair -associated gene group comprising a DNA excision repair protein ERCC1 gene fragment having a nucleotide sequence of SEQ ID NO: 28 and a uracil-DNA glycosylase gene fragment having a nucleotide sequence of SEQ ID NO: 29;
(C) gene fragments selected from a drug resistance-related gene group comprising a topoisomerase 1 gene fragment having a nucleotide sequence of SEQ ID NO: 37, a P-glycoprotein gene fragment having a nucleotide sequence of SEQ ID NO: 38, an equilibrative nucleoside transporter 1 gene fragment having a nucleotide sequence of SEQ ID NO: 39, and a multidrug resistance-associated protein 1 gene fragment having a nucleotide sequence of SEQ ID NO: 40; and
(D) two or more gene fragments selected from a housekeeping gene group comprising a glyceraldehyde-3-phosphate dehydrogenase gene fragment having a nucleotide sequence of SEQ ID NO: 52, a β-actin gene fragment having a nucleotide sequence of SEQ ID NO: 53, and a 40S ribosomal protein S9 gene fragment having a nucleotide sequence of SEQ ID NO: 54.

7. A method for measuring sensitivity of a body fluid specimen or tissue specimen of a cancer patient to an antimetabolite-type anticancer agent or to a combination of such an anticancer agent and another anticancer agent, characterized by comprising hybridizing a DNA array as recited in any of claims 1 to 6 with labeled cDNA probes synthesized through use, as a template, of mRNA obtained from the specimen.


SEQUENCE LISTING (56 sequences)

SEQ ID NO: 1 - DNA, 16 bp, Artificial Sequence
  designed synthetic nucleic acid molecule
tgctggaattcgccct

SEQ ID NO: 2 - DNA, 16 bp, Artificial Sequence
  designed synthetic nucleic acid molecule
ctgcagaattcgccct

SEQ ID NO: 3 - DNA, 368 bp, Artificial Sequence
  designed synthetic nucleic acid molecule
ggatgccgaggtaaaagttctttttgctctaaaagaaaaaggaactaggtcaaaaatctgtccgtgacctatcagttattaatttttaaggatgttgccactggcaaatgtaactgtgccagttctttccataataaaaggctttgagttaactcactgagggtatctgacaatgctgaggttatgaacaaagtgaggagaatgaaatgtatgtgctcttagcaaaaacatgtatgtgcatttcaatcccacgtacttataaagaaggttggtgaatttcacaagctatttttggaatatttttagaatattttaagaatttcacaagctattccctcaaatctgagggagctgagtaacaccatcga

SEQ ID NO: 4 - DNA, 388 bp, Artificial Sequence
  designed synthetic nucleic acid molecule
cgccgagtgttcatcgtcttcagaaaaggctttgttaatataagagctgtccctgaggagatggagcttgctaaggaagaaaagtgtgaatttctgccattcctgtccccacggaaggttatagtaaaaggtgggagaattgttgctatgcagtttgttcggacagagcaagatgaaactggaaaatggaatgaagatgaagatcagatggtccatctgaaagccgatgtggtcatcagtgcctttggttcagttctgagtgatcctaaagtaaaagaagccttgagccctataaaatttaacagatggggtctcccagaagtagatccagaaactatgcaaactagtgaagcatgggtatttgcaggtggtgatgtcgttggtttgg

SEQ ID NO: 5 - DNA, 364 bp, Artificial Sequence
  designed synthetic nucleic acid molecule
ggttttatttctggctcccgagtaagcatgaaaccagaatttcttcacttgactccaggagttcagttggaagcaggaggagataatcttggccaacagtacaatagcccacaagaagttattggcaaacgaggttccgatatcatcattgtaggtcgtggcataatctcagcagctgatcgtctggaagcagcagagatgtacagaaaagctgcttgggaagcgtatttgagtagacttggtgtttgagtgcttcagatacatttttcagatacaatgtgaagacattgaagatatgtggtcctcctgaaagtcactggctggaaataatccaattattcctgcttggattcttccacagggc

SEQ ID NO: 6 - DNA, 308 bp, Artificial Sequence
  designed synthetic nucleic acid molecule
gacaaggtcagcctggtcctcgcacctgccctggcggcatgtggctgcaaggtgccaatgatcagcggacgtggtctggggcacacaggaggcaccttggataagctggagtctattcctggattcaatgtcatccagagcccagagcagatgcaagtgctgctggaccaggcgggctgctgtatcgtgggtcagagtgagcagctggttcctgcggacggaatcctatatgcagccagagatgtgacagccaccgtggacagcctgccactcatcacagcctccattctcagtaagaaactcgtgga

SEQ ID NO: 7 - DNA, 314 bp, Artificial Sequence
  designed synthetic nucleic acid molecule
aagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatgcagccctgccaactgagggacctgcaagggccgcccgctcccttcctgccactgccgcctactggacgctgccctgcatgctgcccagccactccaggaggaagtcgggaggcgtggagggtgaccacaccttggccttctgggaactctcctttgtgtggc

SEQ ID NO: 8 - DNA, 342 bp, Artificial Sequence
  designed synthetic nucleic acid molecule
aaataaacgccatcgccccattggaattggggtacaaggtctggcagatgcttttatcctgatgagatacccttttgagagtgcagaagcccagttactgaataagcagatctttgaaactatttattatggtgctctggaagccagctgtgaccttgccaaggagcagggcccatacgaaacctatgagggctctccagttagcaaaggaattcttcagtatgatatgtggaatgttactcctacagacctatgggactggaaggttctcaaggagaagattgcaaagtatggtataagaaacagtttacttattgccccgatgcctacagcttccactgc

SEQ ID NO: 9 - DNA, 258 bp, Artificial Sequence
  designed synthetic nucleic acid molecule
gggagccaattcacaattcactaagtgactaaagtaagttaaacttgtgtagactaagcatgtaatttttaagttttattttaatgaattaaaatatttgttaaccaactttaaagtcagtcctgtgtatacctagatattagtcagttggtgccagatagaagacaggttgtgtttttatcctgtggcttgtgtagtgtcctgggattctctgccccctctgagtagagtgttgtgggataaaggaatctctcaggg

SEQ ID NO: 10 - DNA, 549 bp, Artificial Sequence
  designed synthetic nucleic acid molecule
cccaacggcggcgagcccttccttataggcgtcagcgggggaacagctagcggcaagtcttccgtgtgtgctaagatcgtgcagctcctggggcagaatgaggtggactatcgccagaagcaggtggtcatcctgagccaggatagcttctaccgtgtccttacctcggagcagaaggccaaagccctgaagggccagttcaactttgaccacccggatgcctttgacaatgaactcattctcaaaacactcaaagaaatcactgaagggaaaacagtccagatccccgtgtatgactttgtctcccattcccggaaggaggagacagttactgtctatcccgcagacgtggtgctctttgaagggatcctggccttctactcccaggaggtacgagacctgttccagatgaagctttttgtggatacagatgcggacacccggctctcacgcagagtattaagggacatcagcgagagaggcagggatcttgagcagattttatctcagtacattacgttcgtcaagcctgcctttgaggaattctgc

SEQ ID NO: 11 - DNA, 384 bp, Artificial Sequence
  designed synthetic nucleic acid molecule
ttctgcagagctgagcgagttcaccacagtggtggggaacaccatgtgcaccttggacttctatgaagggcaaggccgtctggatggggctctctgctcctacacggagaaggacaagcaggcgtatctggaggcagcctatgcagccggcgtccgcaatatcgagatggagtcctcggtgtttgccgccatgtgcagcgcctgcggcctccaagcggccgtggtgtgtgtcaccctcctgaaccgcctggaaggggaccagatcagcagccctcgcaatgtgctcagcgagtaccagcagaggccgcagcggctggtgagctacttcatcaagaagaaactgagcaaggcctgagcgctgccctgcacctccgcagacctgct

SEQ ID NO: 12 - DNA, 302 bp, Artificial Sequence
  designed synthetic nucleic acid molecule
ggcatctgtgctgaacggaccgctatccagaaggccgtctcagaagggtacaaggatttcagggcaattgctatcgccagtgacatgcaagatgattttatctctccatgtggggcctgcaggcaagtcatgagagagtttggcaccaactggcccgtgtacatgaccaagccggatggtacgtatattgtcatgacggtccaggagctgctgccctcctcctttgggcctgaggacctgcagaagactcagtgacagccagagaatgcccactgcctgtaacagccacctggagaacttca

SEQ ID NO: 13 - DNA, 274 bp, Artificial Sequence
  designed synthetic nucleic acid molecule
ttctaaacctgcacttgtccctctccagcaagaggctagcactgaattcattctactcatactacacacccagttatggaatgtccagagttctcgaagaaaataaatgactttaggaagaggtatacattttttaagtcgctctgcctccaaatctgaacagtcactgtaaatcattcttaagcccagatatgagaacttctgctggaaagtgggaccctctgagtgggtggtcagaaaatacccatgctgatgaaatgacctatgcccaaag

SEQ ID NO: 14 - DNA, 254 bp, Artificial Sequence
  designed synthetic nucleic acid molecule
tcgtgccctacctcatagcaggcatccaacacggctgccaggatatcggggcccgcagcctgtctgtccttcggtccatgatgtactcaggagagctcaagtttgagaagcggaccatgtcgccccagattgagggtggtgtccatggcctgcactcttacgaaaagcggctgtactgaggacagcggtggaggccgaggtggtggaggggatgcaccccagtgtccacttttgggcacaggctccctccataa

SEQ ID NO: 15 - DNA, 304 bp, Artificial Sequence
  designed synthetic nucleic acid molecule
tcctggctctcaccacttctctagaagacatgagagagagactgggcaaaatggtggtggcatccagtaagaaaggagagcccgtcagtgccgaagatctgggggtgagtggtgcactgacagtgcttatgaaggacgcaatcaagcccaatctcatgcagacactggagggcactccagtgtttgtccatgctggcccgtttgccaacatcgcacatggcaattcctccatcattgcagaccagatcgcactcaagcttgttggcccagaagggtttgtagtgacggaagcaggatttggagc

SEQ ID NO: 16 - DNA, 331 bp, Artificial Sequence
  designed synthetic nucleic acid molecule
aagagtggacttctcggcccgtactgtcatcacccccgaccccaacctctccattgaccaggttggcgtgccccgctccattgctgccaacatgacctttgcggagattgtcacccccttcaacattgacagacttcaagaactagtgcgcagggggaacagtcagtacccaggcgccaagtacatcatccgagacaatggtgatcgcattgacttgcgtttccaccccaagcccagtgaccttcacctgcagaccggctataaggtggaacggcacatgtgtgatggggacattgttatcttcaaccggcagccaactctgcacaaaatg

SEQ ID NO: 17 - DNA, 285 bp, Artificial Sequence
  designed synthetic nucleic acid molecule
tctaaacctgaaagcatccttgaaatcatgcttgaatattgctttgatagctgctatcatgacccctttttaaggcaattctaatctttcataactacatctcaattagtggctggaaagtacatggtaaaacaaagtaaatttttttatgttctttttttggtcacaggagtagacagtgaattcaggtttaacttcaccttagttatggtgctcaccaaacgaagggtatcagctatttttttttaaattcaaaaagaatatcccttttatagtttgtgcctt

SEQ ID NO: 18 - DNA, 265 bp, Artificial Sequence
  designed synthetic nucleic acid molecule
ccatcggtcaccttgtttctcaactacctcgcatcattgcagatcgtagcgcgttgcctgtcgctttcccttggatacctagaccgttataaagtgtgccacatggacttaccgagcatggagagaggattttagctaggatttgaacacttggtgctgggaacctcagggtattgcttgccactaagccatgaaaccagagacaaaatctctatactgccctgagttggggggaattctcagtgccaactgtggctggtcctca

SEQ ID NO: 19 - DNA, 368 bp, Artificial Sequence
  designed synthetic nucleic acid molecule
cagggtggtggcacattatccctctggggggtggggacgcctgttgttttggctcaatttgggtttgttggtcacatggagctcttccatttcgtttagctgaataatgagttgttcctagaggagacagcctgtctctccttgttgcccccaaagcccatgccctgccgtggtggcagctggggctgtggatgggaggggtccccaacatggatgtgttgcccctcctccgcatgccaacgcagttcatgtacaaggcccctctgcaactggagagaaaattaattcctatcccgtgagtggattgtgagaaattccacccacgtggagacagcttactgcagcactgttggtgttcggagctcttc

SEQ ID NO: 20 - DNA, 422 bp, Artificial Sequence
  designed synthetic nucleic acid molecule
gatctgtgtatagtgacaggtatatttttgcatctaatttgtatgaatctgaatgcatgaatgagacagagtggacaatttatcaagactggcatgactggatgaataaccaatttggccaaagccttgaattggatggaatcatttatcttcaagccactccagagacatgcttacatagaatatatttacggggaagaaatgaagagcaaggcattcctcttgaatatttagagaagcttcattataaacatgaaagctggctcctgcataggacactgaaaaccaacttcgattatcttcaagaggtgcctatcttaacactggatgttaatgaagactttaaagacaaatatgaaagtctggttgaaaaggtcaaagagtttttgagtactttgtgatcttgctgaagactacaggca

SEQ ID NO: 21 - DNA, 322 bp, Artificial Sequence
  designed synthetic nucleic acid molecule
attcagcagaagacccggcttgctccagtgtagctttctacatcccacatcaggtatattagagcttatccgaactggggaaagacggattgagattaactgctgggacctcctacctgcattatctcattctggcttccttgataattctgtgggccttgcagctttaactatagctcagctgctgcaagatttcagacttttgaggatgttgtgtgagggtgtttgactgtgactggggaagctcagactactttgtatgtgaatgcttcagggttttctttgttgagaacaactagcaacaaaggcaacccatgtgtga

SEQ ID NO: 22 - DNA, 391 bp, Artificial Sequence
  designed synthetic nucleic acid molecule
gccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaat

SEQ ID NO: 23 - DNA, 220 bp, Artificial Sequence
  designed synthetic nucleic acid molecule
cgcatgctcaataccctgcagaccaatgccggctacctggagcaggtgaagcgccagcggggtgaccctcagacacagttggaagccatggaactgtacctggcacggagtgggctgcaggtggaggacttggaccggctgaacatcatccacgtcactgggacgaaggggaagggctccacctgtgccttcacggaatgtatcctccgaagctatggcc

SEQ ID NO: 24 - DNA, 364 bp, Artificial Sequence
  designed synthetic nucleic acid molecule
cggtagttgccatggtctgggaggggctgaatgtggtgaagacgggccgagtcatgctcggggagaccaaccctgcagactccaagcctgggaccatccgtggagacttctgcatacaagttggcaggaacattatacatggcagtgattctgtggagagtgcagagaaggagatcggcttgtggtttcaccctgaggaactggtagattacacgagctgtgctcagaactggatctatgaatgacaggagggcagaccacattgcttttcacatccatttcccctccttcccatgggcagaggaccaggctgtaggaaatctagttatttacaggaacttcatcataatttggagggaagctc

SEQ ID NO: 25 - DNA, 372 bp, Artificial Sequence
  designed synthetic nucleic acid molecule
acctgaaagaccgaccattcttccctgggctggtgaagtacatgaactcagggccggttgtggccatggtctgggaggggctgaacgtggtgaagacaggccgagtgatgcttggggagaccaatccagcagattcaaagccaggcaccattcgtggggacttctgcattcaggttggcaggaacatcattcatggcagtgattcagtaaaaagtgctgaaaaagaaatcagcctatggtttaagcctgaagaactggttgactacaagtcttgtgctcatgactgggtctatgaataagaggtggacacaacagcagtctccttcagcacggcgtggtgtgtccctggacacagctcttcattccattgac

SEQ ID NO: 26 - DNA, 406 bp, Artificial Sequence
  designed synthetic nucleic acid molecule
acatctcgcccgtcctgaaggaccccgcctccttccgcgccgccatcggcctcctggcgcgacacctgaaggcgacccacgggggccgcatcgactacatcgcaggcctagactcccgaggcttcctctttggcccctccctggcccaggagcttggactgggctgcgtgctcatccgaaagcgggggaagctgccaggccccactctgtgggcctcctattccctggagtacgggaaggctgagctggagattcagaaagacgccctggagccaggacagagggtggtcgtcgtggatgatctgctggccactggtggaaccatgaacgctgcctgtgagctgctgggccgcctgcaggctgaggtcctggagtgcgtgagcctggtggagctgacctcgcttaa

SEQ ID NO: 27 - DNA, 369 bp, Artificial Sequence
  designed synthetic nucleic acid molecule
ctgcaccgtttattagccagttctacaaggaatcattgatgaaagttatgccttatgttgatatactttttggaaatgagacagaagctgccacttttgctagagagcaaggctttgagactaaagacattaaagagatagccaaaaagacacaagccctgccaaagatgaactcaaagaggcagcgaatcgtgatcttcacccaagggagagatgacactataatggctacagaaagtgaagtcactgcttttgctgtcttggatcaagaccagagagaaattattgataccaatggagctggagatgcatttgttggaggttttctgtctcaactggtctctgacaagccactgactgaatgtatcc

SEQ ID NO: 28 - DNA, 317 bp, Artificial Sequence
  designed synthetic nucleic acid molecule
tgatacccctcgacgaggatgaggtccctcctggagtggccaagcccttattccgatctacacagagccttcccactgtggacacctcggcccaggcggcccctcagacctacgccgaatatgccatctcacagcctctggaaggggctggggccacgtgccccacagggtcagagcccctggcaggagagacgcccaaccaggccctgaaacccggggcaaaatccaacagcatcattgtgagccctcggcagaggggcaatcccgtactgaagttcgtgcgcaacgtgccctgggaatttggcgacgtaattccc

SEQ ID NO: 29 - DNA, 391 bp, Artificial Sequence
  designed synthetic nucleic acid molecule
atctggcccagaaattagggctcaatttcctgattgtagtagaggttaagattgctgtgagctttatcagataagagaccgagagaagtaagctgggtcttgttattccttgggtgttggtggaataagcagtggaatttgaacaaggaagaggagaaaagggaattttgtctttatggggtggggtgattttctcctagggttatgtccagttggggtttttaaggcagcacagactgccaagtactgttttttttaaccgactgaaatcactttgggatattttttcctgcaacactggaaagttttagttttttaagaagtactcatgcagatatatatatatatatttttcccagtcctttttttaagagacggtctttattgggtc

SEQ ID NO: 30 - DNA, 410 bp, Artificial Sequence
  designed synthetic nucleic acid molecule
aaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactaccatacgtctcagggagacccaataggcttaatcctgttgggagaagttgcccttggaaacatgtatgaactgaagcacgcttcacatatcagcaggttacccaagggcaagcacagtgtcaaaggtttgggcaaaactacccctgatccttcagctaacattagtctggatggtgtagacgttcctcttgggaccgggatttcatctggt

SEQ ID NO: 31 - DNA, 309 bp, Artificial Sequence
  designed synthetic nucleic acid molecule
actggcagtgcttccacagccaagaagatagacatcatcaaaggcctctttgtggcctgccgccactcagaagcccggttcatcgctaggtccctgagcggacggctgcgccttgggctggcagagcagtcggtgctggctgccctctcccaggcagtgagcctcacgcccccgggccaagaattcccaccagccatggtggatgctgggaagggcaagacagcagaggccagaaagacgtggctggaggagcaaggcatgatcctgaagcagacgttctgcgaggttcccgacctggaccgaattatc

SEQ ID NO: 32 - DNA, 355 bp, Artificial Sequence
  designed synthetic nucleic acid molecule
caaggtggcccactttaaggactacattccccaggcttttcctgggggccacagcatgatcttggattctgaagtgcttctgattgacaacaagacaggcaaaccactgccctttgggactctgggagtacacaagaaagcagccttccaggatgctaatgtctgcctgtttgtttttgattgtatctactttaatgatgtcagcttgatggacagacctctgtgtgagcggcggaagtttcttcatgacaacatggttgaaattccaaaccggatcatgttctcagaaatgaagcgagtcacaaaagctttggacttggctgacatgataacccgggtgatccaggagggattg

SEQ ID NO: 33 - DNA, 411 bp, Artificial Sequence
  designed synthetic nucleic acid molecule
ggcatctggtaagctcgcatctaaacacctttatataggtggtgatgatgaaccacaagaaaaaaagcggaaagctgccccaaagatgaagaaagttattggaattattgagcacttaaaagcacctaaccttactaacgttaacaaaatttctaatatatttgaagatgtagagttttgtgttatgagtggaacagatagccagccaaagcctgacctggagaacagaattgcagaatttggtggttatatagtacaaaatccaggcccagacacgtactgtgtaattgcagggtctgagaacatcagagtgaaaaacataattttgtcaaataaacatgatgttgtcaagcctgcatggcttttagaatgttttaagaccaaaagctttgtaccatggcagcctcgctt

SEQ ID NO: 34 - DNA, 335 bp, Artificial Sequence
  designed synthetic nucleic acid molecule
cgccatgagcaaacggaaggcgccgcaggagactctcaacgggggaatcaccgacatgctcacagaactcgcaaactttgagaagaacgtgagccaagctatccacaagtacaatgcttacagaaaagcagcatctgttatagcaaaatacccacacaaaataaagagtggagctgaagctaagaaattgcctggagtaggaacaaaaattgctgaaaagattgatgagtttttagcaactggaaaattacgtaaactggaaaagattcggcaggatgatacgagttcatccatcaatttcctgactcgagttagtggcattggtccatctgctg

SEQ ID NO: 35 - DNA, 262 bp, Artificial Sequence
  designed synthetic nucleic acid molecule
ccaccttcatccgtatcatggaccccgacgtgatcaccggttacaacatccagaacttcgaccttccgtacctcatctctcgggcccagaccctcaaggtacaaacattccctttcctgggccgtgtggccggcctttgctccaacatccgggactcttcattccagtccaagcagacgggccggcgggacaccaaggttgtcagcatggtgggccgcgtgcagatggacatgctgcaggtgctgctgcgggagtacaagct

SEQ ID NO: 36 - DNA, 368 bp, Artificial Sequence
  designed synthetic nucleic acid molecule
ctgtcgccatctgttcccaagagacctaaattgccagctccaactcgtaccccagccacagccccagtccctgcccgagcacagggggcagtgacaggcaaaccccgaggagaaggcaccgagcccagacgaccccgagctggcccagaggagctggggaagatccttcagggtgtggtagtggtgctgagtggcttccagaaccccttccgctccgagctgcgagataaggccctagagcttggggccaagtatcggccagactggacccgggacagcacgcacctcatctgtgcctttgccaacacccccaagtacagccaggtcctaggcctgggaggccgcatcgtgcgtaaggagtgggtgct

SEQ ID NO: 37 - DNA, 324 bp, Artificial Sequence
  designed synthetic nucleic acid molecule
tatttcaaagcccagacggaagctcggaaacagatgagcaaggaagagaaactgaaaatcaaagaggagaatgaaaaattactgaaagaatatggattctgtattatggataaccacaaagagaggattgctaacttcaagatagagcctcctggacttttccgtggccgcggcaaccaccccaagatgggcatgctgaagagacgaatcatgcccgaggatataatcatcaactgtagcaaagatgccaaggttccttctcctcctccaggacataagtggaaagaagtccggcatgataacaaggttacttggctggtttcc

SEQ ID NO: 38 - DNA, 265 bp, Artificial Sequence
  designed synthetic nucleic acid molecule
tcagttacccatctcgaaaagaagttaagatcttgaagggtctgaacctgaaggtgcagagtgggcagacggtggccctggttggaaacagtggctgtgggaagagcacaacagtccagctgatgcagaggctctatgaccccacagaggggatggtcagtgttgatggacaggatattaggaccataaatgtaaggtttctacgggaaatcattggtgtggtgagtcaggaacctgtattgtttgccaccacgatagctgaaaa

SEQ ID NO: 39 - DNA, 256 bp, Artificial Sequence
  designed synthetic nucleic acid molecule
tcagccagggaaaaccgagaacaccatcaccatgacaaccagtcaccagcctcaggacagatacaaagctgtctggcttatcttcttcatgctgggtctgggaacgctgctcccgtggaattttttcatgacggccactcagtatttcacaaaccgcctggacatgtcccagaatgtgtccttggtcactgctgaactgagcaaggacgcccaggcgtcagccgcccctgcagcacccttgcctgagcggaactct

SEQ ID NO: 40 - DNA, 399 bp, Artificial Sequence
  designed synthetic nucleic acid molecule
cctgtttgcggtgatctccaggcacagcctcagtgctggcttggtgggcctctcagtgtcttactcattgcaggtcaccacgtacttgaactggctggttcggatgtcatctgaaatggaaaccaacatcgtggccgtggagaggctcaaggagtattcagagactgagaaggaggcgccctggcaaatccaggagacagctccgcccagcagctggccccaggtgggccgagtggaattccggaactactgcctgcgctaccgagaggacctggacttcgttctcaggcacatcaatgtcacgatcaatgggggagaaaaggtcggcatcgtggggcggacgggagctgggaagtcgtccctgaccctgggcttatttcggatcaacgagtctgccga

SEQ ID NO: 41 - DNA, 353 bp, Artificial Sequence
  designed synthetic nucleic acid molecule
gtgctttttgaccacgtaggctgtttaaagaaatatgacacggtgttggatattctaagagacttttttgaactcagacttaaatattatggattaagaaaagaatggctcctaggaatgcttggtgctgaatctgctaaactgaataatcaggctcgctttatcttagagaaaatagatggcaaaataatcattgaaaataagcctaagaaagaattaattaaagttctgattcagaggggatatgattcggatcctgtgaaggcctggaaagaagcccagcaaaaggttccagatgaagaagaaaatgaagagagtgacaacgaaaaggaaactgaaaagagtgactccgt

SEQ ID NO: 42 - DNA, 374 bp, Artificial Sequence
  designed synthetic nucleic acid molecule
ttacgtaaggagtggcttgtgggaatgttgggagcagaatctacaaagcttaacaatcaagcccgtttcattttagagaagatacaagggaaaattactatagagaataggtcaaagaaagatttgattcaaatgttagtccagagaggttatgaatctgacccagtgaaagcctggaaagaagcacaagaaaaggcagcagaagaggatgaaacacaaaaccagcatgatgatagttcctccgattcaggaactccttcaggcccagattttaattatattttaaatatgtctctgtggtctcttactaaagaaaaagttgaagaactgattaaacagagagatgcaaaagggcgagaggtcaatgatcttaa

SEQ ID NO: 43 - DNA, 212 bp, Artificial Sequence
  designed synthetic nucleic acid molecule
ctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaataca

SEQ ID NO: 44 - DNA, 323 bp, Artificial Sequence
  designed synthetic nucleic acid molecule
tgaccagagggttcagagtgggaggcagggccagcccaggccaggagcgcctcatcttcccaggcctcagccacccagggtaaaaggtgccagggaagttgtgggcacctgagaggaggaacagatgtggaggacctgagggtgctcaaagggccaggctcagcctcaagcagtgttttcattgccaacacttactgtacccactccgcagagccccgctgggcctgggccccagggccacagctagcctgcatgtgtgtactgcactttacagtttgcaaagctcttccatacccactctctcaccgaagcctaattgaggc

SEQ ID NO: 45 - DNA, 296 bp, Artificial Sequence
  designed synthetic nucleic acid molecule
cgatgttttcctgtgccctgaggagaccgtaggtgggatcagccctgggaagaccccatcccaggaggtcacttctgaggaggagaacagggccactgactctgccaccatagtgtcaccaccaccatcatctcccccctcatccctcaccacagatcccagccagtctctactcagcctggagcaagaaccgctgttgtcccggatgggcagcctgcgggctcccgtggacgaggaccgcctgtccccgctggtggcggccgactcgctcctggagcatgtgcgggaggacttct

SEQ ID NO: 46 - DNA, 335 bp, Artificial Sequence
  designed synthetic nucleic acid molecule
gctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggg

SEQ ID NO: 47 - DNA, 279 bp, Artificial Sequence
  designed synthetic nucleic acid molecule
cagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgc

SEQ ID NO: 48 - DNA, 368 bp, Artificial Sequence
  designed synthetic nucleic acid molecule
gctgtatcccacggagatcaccgtccatggcaatgggtcctggccctgccgaccacctggagaccttatcaaccctctcaacctcactctttctgaccctggggacaggccatcatccccacagcgcaggcgccgacagctggatccagggggaggccagggccccccacctgtcactctggctgctgccaaaaaagccaagtctgagactgtgctgacctgtgccacagggcgtgcccactgtgtgtggctagagtgccccatccctgatgcccccgttgtcaccaacgtgactgtgaaggcacgagtgtggaacagcaccttcatcgaggattacagagactttgaccgagtccgggtaaatggct

SEQ ID NO: 49 - DNA, 270 bp, Artificial Sequence
  designed synthetic nucleic acid molecule
atctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacg

SEQ ID NO: 50 - DNA, 260 bp, Artificial Sequence
  designed synthetic nucleic acid molecule
gcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttgg

SEQ ID NO: 51 - DNA, 316 bp, Artificial Sequence
  designed synthetic nucleic acid molecule
actccgccaccatgttcgaggcgcgcctggtccagggctccatcctcaagaaggtgttggaggcactcaaggacctcatcaacgaggcctgctgggatattagctccagcggtgtaaacctgcagagcatggactcgtcccacgtctctttggtgcagctcaccctgcggtctgagggcttcgacacctaccgctgcgaccgcaacctggccatgggcgtgaacctcaccagtatgtccaaaatactaaaatgcgccggcaatgaagatatcattacactaagggccgaagataacgcggataccttggcgctagt

SEQ ID NO: 52 - DNA, 333 bp, Artificial Sequence
  designed synthetic nucleic acid molecule
cgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctagacgggaagctcactggcatggccttccgtgtccccactgccaacgtgtcagt

SEQ ID NO: 53 - DNA, 264 bp, Artificial Sequence
  designed synthetic nucleic acid molecule
cattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagta

SEQ ID NO: 54 - DNA, 361 bp, Artificial Sequence
  designed synthetic nucleic acid molecule
tcctgggcctgaagatagaggatttcttagagagacgcctgcagacccaggtcttcaagctgggcttggccaagtccatccaccacgctcgcgtgctgatccgccagcgccatatcagggtccgcaagcaggtggtgaacatcccgtccttcattgtccgcctggattcccagaagcacattgacttctctctgcgctctcctacgggggttggccgcccgggccgcgtgaagaggaagaatgccaagaagggccagggtggggctggggctggagacgacgaggaggaggattaagtccacctgtccctcctgggctgctggattgtctcgttttcctgccaaataaacaggatcagcgc

SEQ ID NO: 55 - DNA, 90 bp, Artificial Sequence
  recombinant vector sequence
caggaaacagctatgaccatgattacgccaagcttggtaccgagctcggatccactagtaacggccgccagtgtgctggaattcgccctt

SEQ ID NO: 56 - DNA, 131 bp, Artificial Sequence
  recombinant vector sequence
agggcgaattctgcagatatccatcacactggcggccgctcgagcatgcatctagagggcccaattcgccctatagtgagtcgtattacaattcactggccgtcgttttacaacgtcgtgactgggaaaac